(19) United States
(12) Patent Application Publication
(10) Pub. No.: US 2008/0044326 A1
(43) Pub. Date: Feb. 21, 2008

(54) Sterilizer for baby products

(75) Inventor: Choong-Sik Shin, Seoul (KR)

Correspondence Address: RABIN & Berdo, PC, 1101 14TH STREET, NW, SUITE 500, WASHINGTON, DC, 20005, US

(73) Assignee: ESENCIA Co., Ltd., Seoul (KR)

(21) Appl. No.: 11/584,531

(22) Filed: Oct. 23, 2006

(30) Foreign Application Priority Data
Jul. 4, 2006 (KR) 20-2006-0019854
Jul. 4, 2006 (KR) 20-2006-0019855

(51) Int. Cl. A61L 2/00

(52) U.S. Cl. 422/300

(57) ABSTRACT

The present invention relates to a sterilizer for baby products that can sterilize, safekeep and sanitizes relatively small-sized baby products and minimize the baby products from being lost. The sterilizer for baby products of the present invention includes: first and second cases (10, 20) each having a storage space therein and a hinge member (12, 22) formed at one edge thereof and into which a shaft is inserted such that the first and second cases (10, 20) are rotatably opened and closed around a shaft; UV generating means provided at any one of the storage spaces of the first and second cases (10, 20); and placing means provided in the storage spaces of the first and second cases (10, 20) to fix the baby products.

### BACKGROUND OF THE INVENTION

1. Field of the Invention

The present invention relates to a sterilizer for baby products that can sterilize and sanitizes relatively small-sized baby products and minimize the baby products from being lost.

2. Description of the Related Art

Generally, since infants or babies have a low immunity, it is required to always sterilize the products used by the infants or babies, for example, nipple, pacifier, device for teeth growth, and a variety of toys, before or after being used.

The baby products and breast feeding supplies are conventionally disinfected by dipping and sterilizing them in a boiling water for a predetermined time and drying them in a natural state. However, the conventional method has a problem in safety that babies may be scalded by the boiling water and a problem that in the natural drying, contaminants contained in the air may be adsorbed on the baby products or breast feeding supplies to cause a secondary infection.

Also, the aforementioned problems can be overcome by keeping the baby products and breast feeding supplies primarily sterilized in a boiling water in a dish dryer having a cover to prevent external contaminants from being penetrated into the dish drier due to the closed cover and thus from being adhered on the baby products or breast feeding supplies. However, the above method also has a problem that the baby products and breast feeding supplies may be secondarily infected by other products dried in the dish drier, for example, dishes for adults.

Meanwhile, when a parent/parents goes/go out of doors with infant and/or babies, it is essentially required to carry the sterilized baby products and break feeding supplies in an amount necessary for the going out time, which increases the volume of the products to carry, thus limiting the parent(s) from going out doors.

In addition, after all the baby products or breast feeding supplies carried for going out of doors have been used, the used baby products or breast feeding supplies have to be again used, which is insanitary.

### SUMMARY OF THE INVENTION

Accordingly, the present invention is directed to a sterilizer for baby products that substantially obviates one or more problems due to limitations and disadvantages of the related art.

An object of the present invention is to provide a sterilizer for baby products that can keep the baby products and breast feeding supplies simply, conveniently and sanitarily by including first and second cases provided with UV generating means disposed at one side thereof and a storage space formed therein to keep the baby products and breast feeding supplies therein, placing means disposed in any one of the first and second cases, and coupling means to which a neck of a bottle is coupled at any one of the first and second cases, wherein the baby products and breast feeding supplies, such as a nursing bottle, are coupled and fixed to the coupling means in the storage space of the first and second cases in a home or in particular when a parent/parents goes/go out of doors, and the baby products including the nursing bottle are sterilized by ultraviolet rays radiated from the UV generating means.

Additional advantages, objects, and features of the invention will be set forth in part in the description which follows and in part will become apparent to those having ordinary skill in the art upon examination of the following or may be learned from practice of the invention. The objectives and other advantages of the invention may be realized and attained by the structure particularly-pointed out in the written description and claims hereof as well as the appended drawings.

To achieve these objects and other advantages and in accordance with the purpose of the invention, as embodied and broadly described herein, there is provided a sterilizer for baby products, comprising: first and second cases each having a storage space therein and a hinge member formed at one edge thereof and into which a shaft is inserted such that the first and second cases are rotatably opened and closed around a shaft; UV generating means provided at any one of the storage spaces of the first and second cases; and placing means provided in the storage spaces of the first and second cases to fix the baby products.

In another aspect of the present invention, there is provided a sterilizer for baby products, comprising: first and second cases each having a storage space therein and a hinge member formed at one edge thereof and into which a shaft is inserted such that the first and second cases are rotatably opened and closed around a shaft; UV generating means provided at any one of the storage spaces of the first and second cases; and a fixing member having at least one coupling hole formed at any one of the first and second cases and communicating with the storage space, for fixing a neck of a bottle through the coupling hole and sterilizing an inside of the bottle of which the neck is fixed.

It is to be understood that both the foregoing general description and the following detailed description of the present invention are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this application, illustrate embodiment(s) of the invention and together with the description serve to explain the principle of the invention. In the drawings:

FIG. 1 is a partial disassembled perspective view of a sterilizer for baby products according to a first embodiment of the present invention;

FIG. 2 is a sectional view of FIG. 1;

FIG. 3 is a perspective view showing that a nipple is placed in the sterilizer for baby products according to a first embodiment of the present invention;

FIG. 4 is a perspective view showing that a pacifier is placed in the sterilizer for baby products according to a first embodiment of the present invention;

FIG. 5 is a partial disassembled perspective view of placing means according to a second embodiment of the present invention;

FIG. 6 is a sectional view showing a usage of the placing means according to the second embodiment of the present invention;

FIG. 7 is a perspective view showing a usage of the placing means according to a third embodiment of the present invention;

FIG. 8 is a perspective view showing that the placing means is separated from a case according to a fourth embodiment of the present invention;

FIG. 9 is a disassembled perspective view of a sterilizer for baby products according to a fifth embodiment of the present invention;

FIG. 10 is a sectional view of FIG. 9;

FIG. 11 is a block diagram showing a constitution of a single ended electronic discharge tube On/Off circuit according to the present invention; and

FIG. 12 is a detailed circuit diagram of a single ended electronic discharge tube On/Off circuit according to the present invention.

### DETAILED DESCRIPTION OF THE INVENTION

Reference will be made in detail to the preferred embodiments of the present invention, examples of which are illustrated in the accompanying drawings.

### First Embodiment

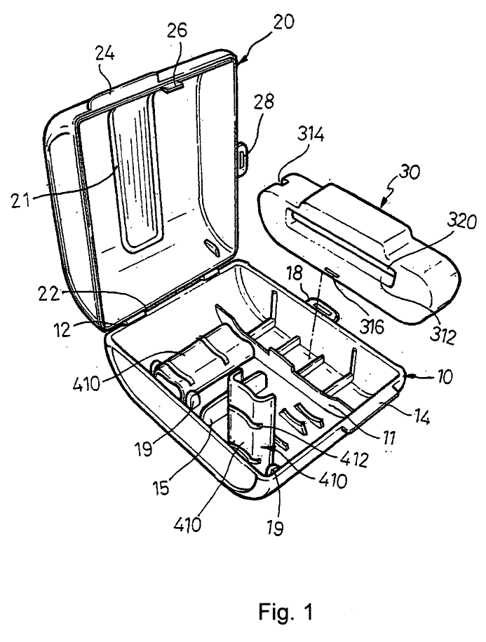

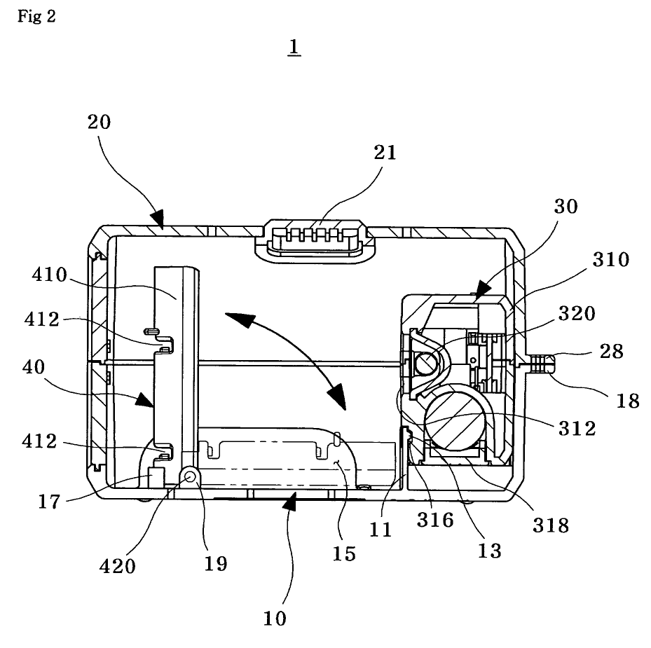

FIG. 1 is a partial disassembled perspective view of a sterilizer for baby products according to a first embodiment of the present invention, and FIG. 2 is a sectional view of FIG. 1.

Referring to FIGS. 1 and 2, a sterilizer 1 for baby products includes first and second cases 10 and 20 each having a storage space therein and that can be relatively opened and closed, UV generating means 30 provided in the storage spaces of the first and second cases 10 and 20, and placing means 40.

The UV generating means 30 and the placing means 40 can be disposed in the first case 10 and a window 21 for viewing an inside of the second case can be formed of a transparent material in the second case 20. However, it is preferable that the window 21 is formed of a semi-transparent material such that light is transmitted by half of light emitting when a sterilization lamp of the UV generating means 30 is turned on to amplify the design of the baby products using a light scattering.

Each of the first and second cases 10 and 20 has a rotational member 12, 22 formed at one end, into which a hinge shaft is inserted such that the first and second cases 10 and 20 can rotate around the hinge shaft, and a protrusion piece 14, 24 formed at the other end thereof.

The first and second cases 10 and 20 have latch means for preventing the first and second cases 10 and 20 from being arbitrarily rotated. The latch means includes a latch hole formed at any one of the first and second cases 10 and 20, and a latch jaw formed at a position corresponding to the latch hole. In this embodiment, the latch hole 16 is formed in the first case 10, and the latch jaw 26 is formed in the second case 20.

Of course, the latch means can be implemented in various ways in addition to the way using the latch hole and the latch jaw. In other words, any means will be possible if it can prevent the first and second cases 10 and 20 from being arbitrarily opened.

Either of the first case 10 or the second case 20 has a loop member 18, 28 to which a string or the like is coupled for convenient carrying.

A barrier 11 for forming a space into which the UV generating means 30 is inserted between an outer wall and the storage space is disposed in the storage space of the first case 10. At least one latch jaw 13 is formed on the barrier 11 and the outer wall, and a latch hole 316 is formed in the UV generating means 30 at a position corresponding to the latch jaw 13.

A plurality of baby products are placed in the storage space of the first case 10 through the placing means 40, and a separation wall 15 for preventing the placed baby products from contacting one another is formed in the storage space of the first case 10.

A pushing protrusion 23 protruded downward is formed in the storage space of the second case 2. The pushing protrusion 23 pushes a switch of the UV generating means 30 disposed in the storage space of the first case 10 to operate a sterilizing lamp when the first case 10 and the second case 20 are closed each other centering on the rotational member 12, 22.

It is preferable that a plurality of drain holes are formed in each of the first and second cases 10 and 20. The drain holes formed in the first case 10 drain water extracted from the baby products, and the drain holes formed in the second case 20 prevents moisture from being generated from the inside of the first and second cases 10 and 20 while moisture generated from the baby products sterilized through the sterilizing lamp of the UV generating means 30 is drained to the outside.

The UV generating means 30 includes a main body 310 having a through hole 312 formed at a front surface thereof and a radiation hole 314 formed at an upper portion of a position corresponding to the pushing protrusion 23 of the second case 20, a sterilizing lamp 320 stored within the main body 310 and radiating UV through the radiation hole, a UV generating part stored within the main body 310 and having a circuitry transmitting a control signal for controlling the sterilizing lamp 320, and a switch disposed at a predetermined portion of the main body 310 and switched by a pushing operation of the pushing protrusion 23 of the second case 20 inserted through the radiation hole 314, for transmitting the same to the UV generating part.

An opening and closing plate 318 is formed in the main body 310 such that a battery can be easily installed.

Preferably, the switch is comprised of a limit switch. In this case, an operation pin of the limit switch is preferably formed in a depressed state without being protruded to an outside in the downward direction of the radiation hole 314 of the main body 310.

Of course, the switch is not limited to only the limit switch. All means will be possible if it can transmit a control signal capable of operating the sterilizing lamp 320 by a closing operation of the second case 20. For example, the switch may be comprised of a reed switch.

The placing means 40 includes a placing bar 410, and hinges 420 protruded from both sides of the placing bar 410. A hinge member 19 is formed at a bottom of the first case 10 integrally with the first case to support the hinge 420 such that the hinge 420 is inserted and rotated.

When the placing bar 410 is rotated around the hinge 420 and thus stands erect, a supporting bar 17 for supporting the standing of the placing bar 410 is formed at a predetermined portion of the first case 10.

In this case, it is preferable that fixing means is formed in the placing bar 410 and the supporting bar 17. For example, a latch groove is formed in the latch bar 410 and a latch hook latched in the latch groove is formed at a predetermined portion of the supporting bar 17, so that the placing bar 410 is rigidly fixed by the supporting bar 17 to prevent the placing bar 410 from being arbitrarily rotated due to an impact.

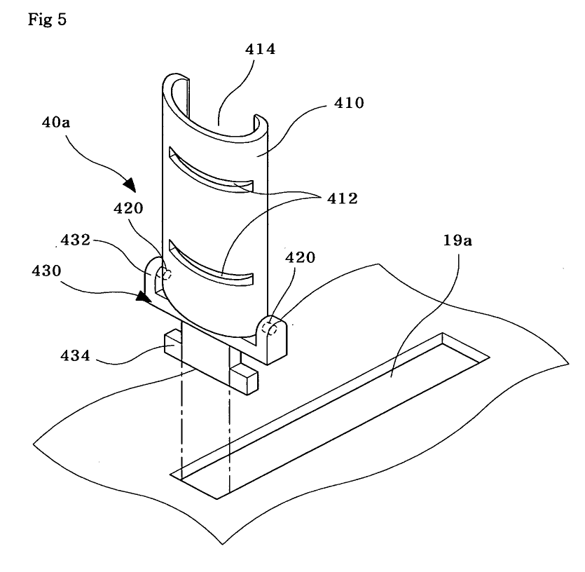

Referring to FIG. 5, the placing bar 410 is configured to have both side ends bent inward to form a latch space 414, and at least one placing slot 412 in a lengthwise direction.

Use of the aforementioned sterilizer for baby products will now be described.

First, when the protrusion pieces 14 and 24 formed in the first and second cases 10 and 20 are pushed each other, the first and second cases 10 and 20 are opened while being rotated centering on the hinge shaft inserted into the rotational members 12 and 22 of the first and second cases 10 and 20.

Baby products are placed through the placing means 40 formed in the storage spaces of the first and second cases 10 and 20 opened each other. Hereinafter, placing a nipple will be described as one example.

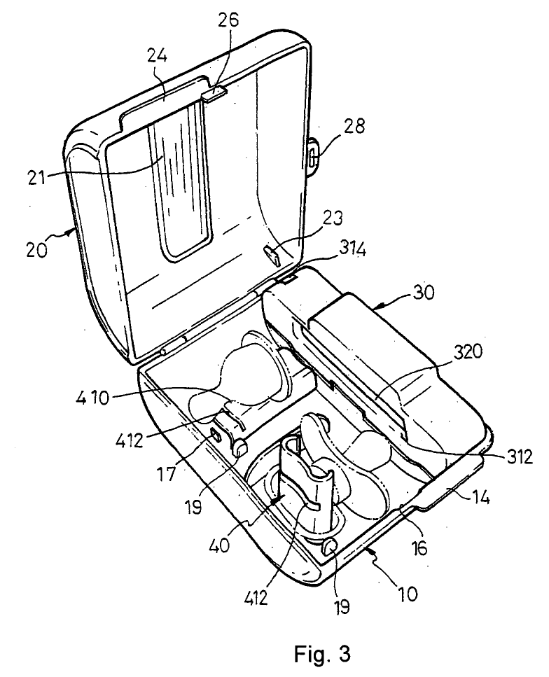

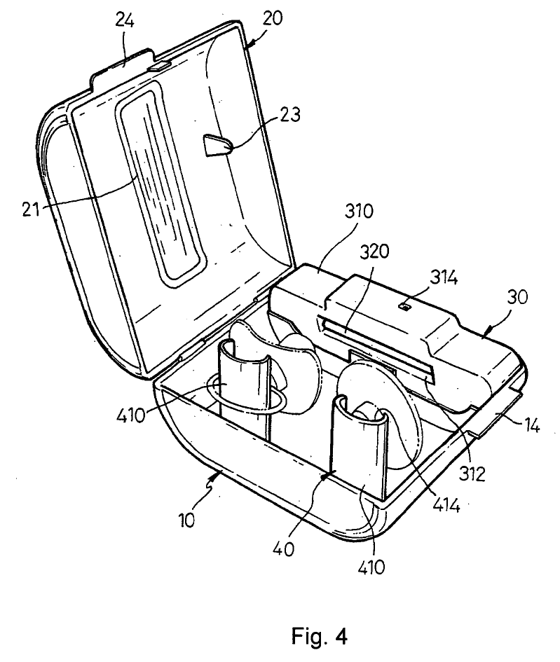

FIG. 3 is a perspective view showing that a nipple is placed in the sterilizer for baby products according to a first embodiment of the present invention, and FIG. 4 is a perspective view showing that a pacifier is placed in the sterilizer for baby products according to a first embodiment of the present invention. FIGS. 3 and 4, the placing bar 410 of the placing means 40 is rotated centering on the hinge 420 and is laid to be parallel with a bottom surface of the first case 10.

By doing so, the placing slot 412 is directed toward an upper direction and then one end of a nipple is inserted into the placing slot 412 and fixed.

The pacifiers are generally classified into ring type pacifiers having a gripping ring formed at a rear end thereof and protrusion type pacifiers having a protrusion piece formed at a rear end thereof. In the case of the ring type pacifier, the pacing bar 410 of the placing means 40 stands erect centering on the hinge 420 and the ring of the pacifier is inserted into the placing slot 412 of the placing bar 410 and is then fixed.

In addition, in case where the placing bar is erected perpendicularly, the placing bar 410 is erected perpendicularly through the supporting bar 17 provided in the first case 10, and a further rotation is prevented by the fixing means, so that the placing bar 410 is rigidly erected.

Meanwhile, in the case of the protrusion type pacifier, the placing bar 410 is erected perpendicularly and the protrusion piece of the protrusion type pacifier is inserted into and fixed to the placing space 414 formed inside the placing bar 410.

Meanwhile, if the erection of the pacifier is ended through the placing means 40, the first and second cases 10 and 20 are closed.

At this time, when the first and second cases 10 and 20 are closed, the pushing protrusion 23 protruded from the inside of the second case 20 is inserted into the radiation hole 314 formed in the main body 310 of the UV generating means 30 of the first case 10, and the operation pin of the switch depressed in the radiation hole 314 is pressed by the pushing protrusion 23.

The switch of the pushed operation pin transmits a pushing signal, i.e., ON signal, to the circuitry, and the circuitry receiving the ON signal turns On the sterilizing lamp to sterilize the baby products.

Also, since the first and second cases 10 and 20 are in the closed state, the UV radiated from the sterilizing lamp 320 is not leaked to an outside. Especially, the sterilizing lamp 320 is turned ON only during a time set in the circuitry.

Further, On/Off of the sterilizing lamp 320 can be confirmed through the window 21 of the second case 20. Furthermore, the light of the sterilizing lamp is scattered to amplify the design effect.

Accordingly, the sterilizer for baby products of the present invention enables to always provide the sterilized baby products regardless of places.

### Second Embodiment

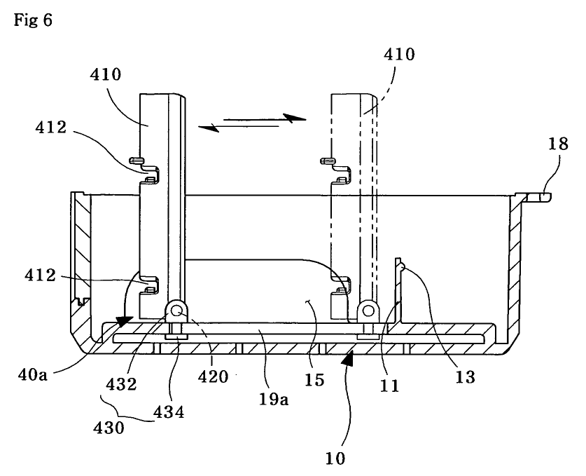

FIG. 5 is a partial disassembled perspective view of placing means according to a second embodiment of the present invention, and FIG. 6 is a sectional view showing a usage of the placing means according to the second embodiment of the present invention. Referring to FIGS. 5 and 6, unlike in the first embodiment that described the placing means fixed in the first case, in the second embodiment, the placing means is a sliding means that slides along the floor of the first case. Like reference numerals are used to denote like elements described in the First Embodiment, and repetition of detailed descriptions of these elements are omitted herefrom.

The sliding type placing means 40a includes a placing bar 410 forming a placing space 414 within, a plurality of placing slots 412 formed in a lengthwise direction, and hinge protrusions 420 protruding from either side thereof. A sliding member 430 includes hinge receptacles 432 provided at an upper portion thereof for respectively receiving the hinge protrusions 420 that are inserted therein, and a sliding member 434 formed at the lower portion thereof.

The sliding member 434 has a stepped recess formed a predetermined depth into either side thereof, to latch onto either side of the sliding slot 19a of the first case 10.

The first case 10 with the placing means 40a formed therein, forms the sliding slot 19a in which the sliding member 434 inserts and slides along.

To prevent the intrusion of foreign objects through the sliding slot 19a, the first case 10 may be formed in a double panel configuration. In this case, the sliding slot 19a is formed in the inner panel of the first case 10, and a plurality of drain holes are formed in the outer panel.

To use the above-configured sliding type placing means 40a, the sliding member 434 is inserted so that either side of the sliding slot 19a of the first case 10 latches respectively into the stepped recesses of the sliding member 434.

Moreover, the hinge protrusions 420 are formed on the placing bar 410, to be inserted into the hinge receptacles 432 of the sliding member 430, enabling the placing bar 410 to be pivoted.

Accordingly, the placing bar 410 can be slid along the sliding slot 19a of the first case 10, enabling a variety of baby products to be placed on the placing bar 410, and increasing storage effectiveness of the storage spaces of the first and second cases 10 and 20.

Although the placing bar 410 may be made to pivot on a hinge, the hinge may be omitted, and the placing bar 410 may be formed integrally with the sliding member 430, as dictated by the application of the product.

### Third Embodiment

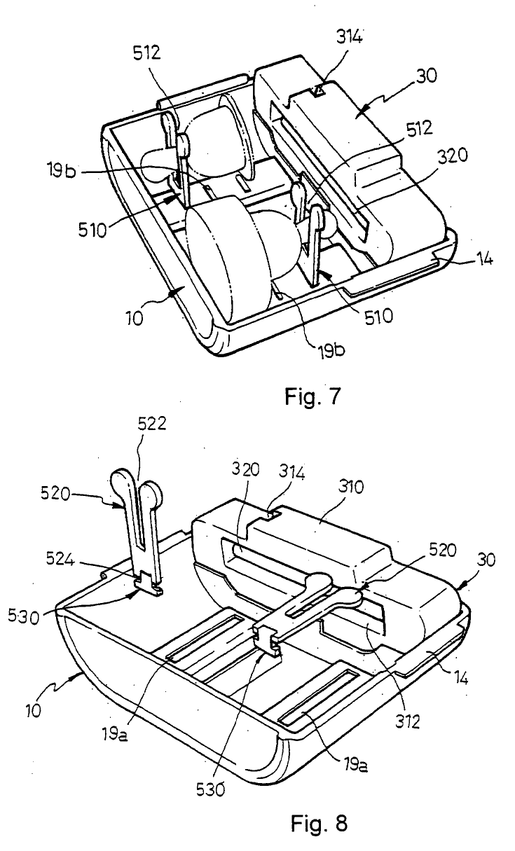

FIG. 7 is a perspective view showing a usage of the placing means according to a third embodiment of the present invention. Like reference numerals are used to denote like elements described in the First Embodiment, and repetitions of detailed descriptions of these elements are omitted herefrom.

Referring to FIG. 7, a placing means according to the third embodiment of the present invention includes a placing bar 510 with a gripping recess 512 recessed a predetermined distance in a central, upper portion thereof, and a coupling protrusion (not shown) protruding at the bottom of the placing bar 510.

Also, an insertion slot 19b is formed in a lengthwise direction along the floor of the first case 10, allowing the coupling protrusion (not shown) of the placing bar 510 to be detachably inserted therein.

Accordingly, a placing bar 510 of the placing means may be erected by inserting the coupling protrusion protruding from the lower portion thereof into any one of the plurality of insertion slots 19b formed in the first case 10, and a baby product may be fixed with the gripping recess 512 formed in the upper portion of the placing bar 510.

### Fourth Embodiment

FIG. 8 is a perspective view showing the placing means separated from a case according to a fourth embodiment of the present invention. Like reference numerals are used to denote like elements described in the First and Second embodiments, and repetitions of detailed descriptions of these elements are omitted herefrom.

Referring to FIG. 8, a sliding type placing means according to the fourth embodiment includes a placing bar 520, and a sliding member 530 provided at the lower end of the placing bar 520.

The placing bar 520 has a gripping recess 522 recessed a predetermined distance from an upper central portion thereof downward, and a storage recess 524 forming a space of a predetermined depth at a lower portion thereof.

The sliding member 530 forms a protruding part that is partially inserted into the storage recess 524. A protruding portion is formed downward from the protruding part. When the protruding part partially inserts into the storage recess 524, a predetermined sliding space is formed between the lower portion of the placing bar 520 and the protruding portion.

Of course, a hinge is formed between the protruding part of the sliding member 530 and the storage recess 524, to enable the placing bar 520 to pivot.

Here, the sliding slot 19a is formed in the first case 10.

Thus, the sliding slot 19a of the first case 10 is inserted through the space between the lower portion of the placing bar 520 and the protruding portion, so that the placing bar 520 can slide along the sliding slot 19a.

### Fifth Embodiment

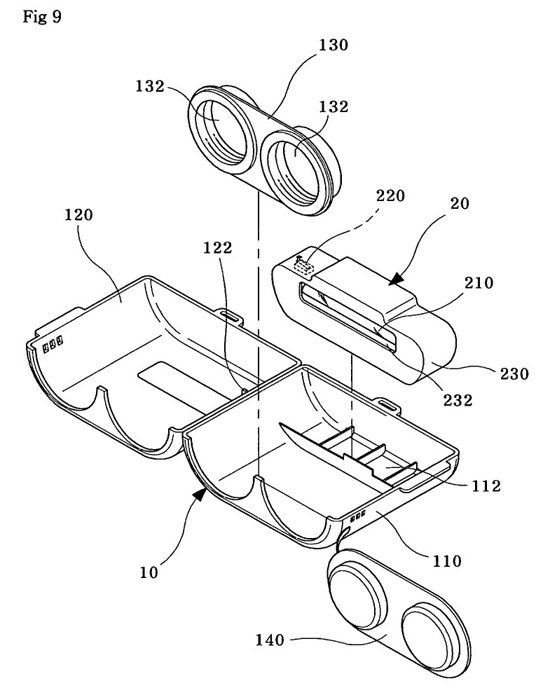

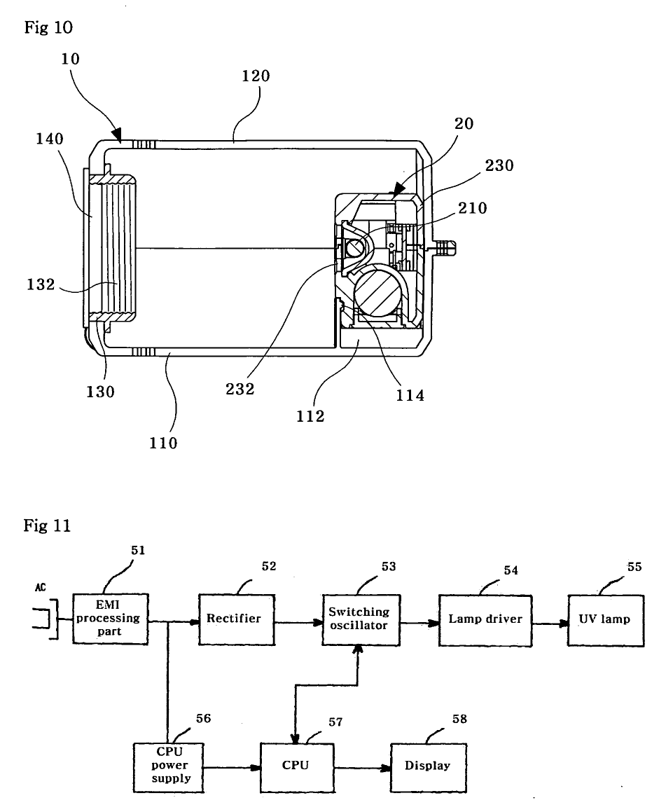

FIG. 9 is a disassembled perspective view of a sterilizer for baby products according to a fifth embodiment of the present invention. Like reference numerals are used to denote like elements described in the First Embodiment, and repetitions of detailed descriptions of these elements are omitted herefrom.

Referring to FIG. 9, a baby product sterilizer includes first and second cases 10 and 20, an ultraviolet (UV) generator 30 for emitting ultraviolet rays disposed within the first and second cases 10 and 20, and a fixing member 610 disposed on one side of the first and second cases 10 and 20, for fixing bottlenecks.

The seating recesses are formed on one side of the first and second cases 10 and 20 to receive and removably fix the fixing member 610 therein.

A finishing cap 620 is provided to seal coupling holes 612 formed in the fixing member 610 on one side of the first and second cases 10 and 20.

The fixing member 610 may be formed in a position opposite to the UV generating means 30. At least one or more coupling holes 612 for inserting bottle necks are provided, and the coupling holes 612 have female screw threads formed therein, in which male screw threads formed on bottle necks may be fastened.

The finishing cap 620 may be in any form that is capable of sealing the coupling holes 612 of the fixing member 610. Also, the finishing cap 620 forms an eyelet on one side thereof through which a string may be fastened, so that the finishing cap 620 may be connected to any side of the first and second cases 10 and 20 via the string. The finishing cap 620 may be formed of any sealing material such as rubber or silicon.

The fixing member 610 may be formed to be removably attachable to the first and second cases 10 and 20. However, it may also be integrally formed with the first case 110 on one side thereof.

The fixing member 610 is not limited to being fixable to baby feeding bottles, but may also be used to sterilize interiors of drink bottles and recyclable bottles. The fixing member 610 may be formed in different sizes to accommodate the bottle necks of various types of bottles for sterilization and drying, and may be interchangeably attached to the first case 10.

A detection sensor may be further provided within the first and second cases in the first through fifth embodiments, whereby the detection sensor detects whether baby products are stored in the storage spaces of the first and second cases or if a bottle has been coupled to the coupling holes. Such a detection sensor would prevent random emission of ultraviolet rays, so that unnecessary power consumption due to unneeded ultraviolet emission is prevented.

Furthermore, the detection sensor used may selectively be a contact-type or a non-contact-type sensor. A contact-type detection sensor may include a limit switch protrudingly formed on an inner surface of the first and second cases, so that when the switch is pressed, the baby products stored within the cases are detected.

A non-contact-type detection sensor is an infrared sensor that includes an emitter for emitting infrared rays on a side thereof, a receiver formed on the other side for receiving the emitted infrared rays. When the path of an infrared beam between the emitter and the receiver is blocked within the storage of the sterilizer, the detection sensor detects the presence of a baby product in the storage space of the cases.

Moreover, after a first sensing determining whether a baby product is stored inside the storage space of the first and second cases through the contact or non-contact-type detection sensor, the UV generating means emits ultraviolet rays to sterilize products for sterilization through a second sensing, when the first and second cases are closed together.

When the second sensing is not performed due to the first and second cases being open after the detection sensor performs the first sensing, or when the first sensing is not performed due to a lack of baby products, if the first and second cases are closed causing the second sensing to be performed, the UV generator does not emit ultraviolet rays.

That is, the UV generator emits ultraviolet rays only when the conditions for both the first sensing and the second sensing are met.

The use of the above-described contact-type or non-contact-type sensors is but one embodiment, to which the present invention is not limited. Alternate means for determining whether baby products are present in the storage spaces of the first and second cases may be used.

In the first through fifth embodiments, the present invention is not limited to having a UV generating means as a sterilizing means, but may alternately employ an ozone generator that emits O₃ for sterilizing and eliminating odors. Such an ozone generator may be detachably disposed within the first and second cases.

The ozone generator may selectively be a commercial frequency-type or mid frequency-type generator.

Of course, the present invention is not limited to ultraviolet or ozone sterilization, and may employ a variety of sterilizing means according the required characteristics of the sterilizer.

In the first through fifth embodiments, the sterilizing lamp 320 of the UV generating means 30 may operate for a predetermined duration of approximately 5-6 minutes in a first sterilizing process when the first and second cases 10 and 20 are closed together, and may operate in a second sterilizing process after a certain duration has elapsed from the end of the first sterilizing process. The first and second sterilizing processes may be made to repeat several times in accordance with sterilizing programming.

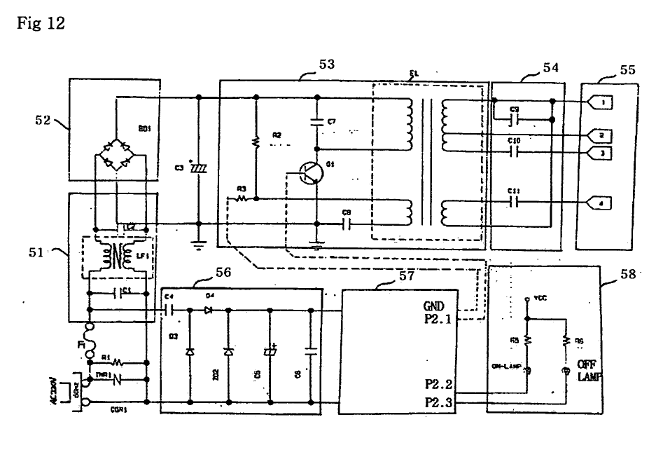

FIG. 11 is a block diagram of a single-ended type electronic discharging and lighting circuit according to the present invention. 51 is an EMI processing part, 52 is a rectifier supplying an input voltage to a switching oscillator, 53 is a switching oscillator, 54 is a lamp driver, and 55 is a sterilizing lamp. Also, 56 is a CPU power supply that supplies power for a CPU, 57 is a CPU that links and operates the switching oscillator 53 and controls the circuit, and 58 is an operation indicator.

Below, a detailed description of a single-ended type electronic discharging and lighting circuit according to the present invention will be given, with reference to FIG. 11.

First, when power (for example, AC 220V) is inputted, the inputted power voltage is provided to the EMI processing part 51. The EMI processing part 51 removes an electrostatic discharge generated by the inputted power and supplies the voltage to the rectifier 52 through a line filter (not shown).

Next, the rectifier 52 rectifies the inputted power voltage and provides a stable voltage to the switching oscillator 53 for driving the sterilizing lamp 55. The switching oscillator 53 links the input power provided by the rectifier 52 with the controlling of the CPU 57, and performs an oscillation for driving the sterilizing lamp 55. Here, the switching oscillator 53 according to the present invention is a single ended-type device with a circuit that uses a single transistor to perform oscillation.

The switching oscillator 53 provides an oscillation voltage to the lamp driver 54, and the lamp driver 54 receives the oscillation voltage from the switching oscillator 53 to activate or deactivate the sterilizing lamp 55.

The EMI processing part 51 supplies power to the CPU power supply 56 through a voltage current limiting capacitor (not shown).

The CPU power supply 56 employs the voltage current limiting capacitor (C4 in FIG. 12) to amplify the input voltage, and generates a predetermined voltage for driving the CPU 57, through a diode for preventing instantaneous voltage surges, a half-wave rectifier, etc. In the present embodiment, a constant potential voltage of approx. 1.8V-3.6V is generated for providing to the CPU 57.

The CPU 57 is supplied with a predetermined stable voltage from the CPU power supply 56. The CPU 57 controls a predetermined signal using a built-in oscillator (not shown) and outputs the controlled signal to the switching oscillator 53. In order to reduce a manufacturing cost, a very cheap commercial CPU was used herein. As an example, when an integrated circuit (IC) for a domestic remote controller is used, a manufacturing cost of the entire circuit can be greatly reduced.

In the EMI processing part 51, capacitors C1 and C2 and a resistor R1 connected before and after a line filter LF1 are used for removing harmful electromagnetic waves from the input signal. It can be seen from Equation 1 that the rectifier 52 outputs about DC 280 when the input voltage is AC 220V. In the switching oscillator 53, a capacitor C7 connected to a collector of a transistor Q1 is a snubber capacitor that prevents active elements of a related circuit from being damaged by a surge voltage of an inductive load when the input voltage abruptly rises or changes. When a control voltage is applied to a base (B) of the transistor Q1 under control of the CPU 57, the transistor Q1 outputs a signal for driving the ultraviolet lamp 55 to the lamp driver 54.

Although not shown in detail, the ultraviolet lamp 55 includes a filament connected between a port 1 and a port 2 and a filament connected between a port 3 and a port 4. Thus, the ultraviolet lamp 55 operates when a turn-on signal is applied thereto.

A capacitor C4 disposed at an input side of the CPU power supply 56 functions to intensify the input voltage. Also, a diode D3 and a zener diode ZD2 construct a half-wave rectifier circuit, and a diode D4 protects against instantaneous overvoltage.

The input voltage that is current-limited by the elements R1 and C2 is half-wave rectified through the diodes D3 and D4. Then, the half-wave-rectified voltage is voltage-regulated by the zener diode ZD2 and then is stored in the capacitor C6.

According to the present invention, a manufacturing cost of the sterilizer can be greatly reduced by using the circuit applying a low current. Therefore, the apparatus according to the present invention can be practically used in general homes or hospitals.

Also, the CPU provides the active signal using the oscillation frequency (in this embodiment, 2.85 MHz) of the oscillator as the basic clock, thereby preventing the circuit from operating arbitrarily.

When the ultraviolet lamp is turned on, the red LED of the operation indicator 58 is turned on, thus providing convenience to the users. The green LED is configured to indicate the standby state. Thus, the users can always check the operation state of the circuit.

Moreover, the reset terminal of the CPU 57 is connected to the door switch (not shown) of the sterilizer. Therefore, when the user opens the door, he/she can be protected from the emission of the sterilization light energy of the ultraviolet lamp, thereby guaranteeing the safety.

The apparatus according to the present invention provides a UV generating means on one side thereof, and a first and second case having placing means within for placing baby products thereon. Baby products may be stored in the storage space formed by the first and second cases in the home, and especially during outings. Ultraviolet rays emitted by the UV generating means sterilize the stored baby products, so that baby products can be maintained in a constantly sanitary state, regardless of time or place.

Moreover, a fixing member for fixing bottles is formed on a side of the first and second cases that have the UV generating means stored within, so that the insides of the bottles fixed by the fixing member may be sterilized and dried by the ultraviolet rays emitted by the UV generator. This function can be applied to a variety of baby products.

It will be apparent to those skilled in the art that various modifications and variations can be made in the present invention. Thus, it is intended that the present invention covers the modifications and variations of this invention provided they come within the scope of the appended claims and their equivalents.

What is claimed is:

1. A sterilizer for baby products, comprising:
first and second cases (10, 20) each having a storage space therein and a hinge member (12, 22) formed at one edge thereof and into which a shaft is inserted such that the first and second cases (10, 20) are rotatably opened and closed around a shaft;
UV generating means provided at any one of the storage spaces of the first and second cases (10, 20); and
placing means provided in the storage spaces of the first and second cases (10, 20) to fix the baby products.

2. The sterilizer of claim 1, wherein any one of the first and second cases (10, 20) comprises a pushing protrusion (23) for pushing a control switch of the UV generating means 30.

3. The sterilizer of claim 1, wherein the first and second cases (10, 20) comprise a plurality of drain holes for communicating with the external air and draining moisture.

4. The sterilizer of claim 1, wherein the UV generating means (30) comprises:
a UV lamp (320);
a controller controlling operations of a power supply, a switch determining whether the first and second cases (10, 20) are opened or closed, and the UV lamp (320) using a power of the power supply depending on the operation of the switch; and
a main body including the UV lamp and the controller, the main body having a radiation hole (312) formed at one side thereof, for radiating UV of the UV lamp (320) to an outside, and a through hole (314) formed at an upper portion where the switch of the controller is positioned and communicating with the outside,
wherein the main body (310) is configured to be attachable and detachable in the storage spaces of the first and second cases (10, 20).

5. The sterilizer of claim 1, wherein the UV generating means (30) comprises a single ended electronic discharge tube On/Off circuit.

6. The sterilizer of claim 1, wherein the placing means (40) comprises:
a placing bar (410); and
a hinge (420) protruded from both sides of a lower portion of the placing bar (410),
wherein any one of the first and second cases (10, 20) comprises a hinge member (19) formed at a bottom thereof, for supporting a hinge (420) of the placing bar (410).

7. The sterilizer of claim 6, wherein the placing bar (410) has a plane section shape of which one end is opened, both ends of the opened one end being bent inward to form a hanging space (414) therein.

8. The sterilizer of claim 6, wherein the placing bar (410) comprises a plurality of placing slots (412) formed at one side of the placing bar (410) in a lengthwise direction of the placing bar (410) and having a predetermined depth.

9. The sterilizer of claim 1, wherein the placing means (40a) comprises a placing bar (410), a hinge (420) protruded from both sides of a lower portion of the placing bar (410), a rotational member (432) rotatably supporting the hinge (420), and sliding means (430) having a sliding member (434) formed at a lower portion of the placing means (40a), the placing bar (410), the hinge (420) and the rotational member (432) being formed at an upper portion of the placing means (40a).

10. The sterilizer of claim 1, wherein the placing means comprises a placing bar (510) having a gripping recess (512) formed a predetermined depth in a downward direction of a center of an upper portion, and a coupling protrusion protruded from a lower portion of the placing bar (510),
wherein any one of the first and second cases (10, 20) comprises at least one insertion hole (19b) formed at a bottom thereof, for fixedly coupling the coupling protrusion.

11. The sterilizer of claim 1, wherein the placing means comprises a placing bar (510) having a gripping recess (512) formed a predetermined depth in a downward direction of a center of an upper portion and a storage recess (524) formed a predetermined depth at a lower portion thereof, and sliding means (430) having a protrusion piece partially inserted into the storage recess (524) of the placing bar (520) and protrusion parts formed at the storage recess (524), coupled to one another to be rotatable by a hinge, and formed at a predetermined interval at a lower portion of the placing bar 520,
wherein any one of the first and second cases (10, 20) comprises a sliding slot (19a) into which an interval between the placing bar (520) and the protrusion parts is inserted.

12. A sterilizer for baby products, comprising:
first and second cases (10, 20) each having a storage space therein and a hinge member (12, 22) formed at one edge thereof and into which a shaft is inserted such that the first and second cases (10, 20) are rotatably opened and closed around a shaft;
UV generating means provided at any one of the storage spaces of the first and second cases (10, 20); and
a fixing member (610) having at least one coupling hole (612) formed at any one of the first and second cases (10, 20) and communicating with the storage space, for fixing a neck of a bottle through the coupling hole (612) and sterilizing an inside of the bottle of which the neck is fixed.

13. The sterilizer of claim 12, wherein any one of the first and second cases (10, 20) comprises a pushing protrusion (23) for pushing a control switch of the UV generating means 30.

14. The sterilizer of claim 12, wherein the first and second cases (10, 20) comprise a plurality of drain holes for communicating with the external air and draining moisture.

15. The sterilizer of claim 12, wherein the UV generating means (30) comprises:
a UV lamp (320);
a controller controlling operations of a power supply, a switch determining whether the first and second cases (10, 20) are opened or closed, and the UV lamp (320) using a power of the power supply depending on the operation of the switch; and
a main body including the UV lamp and the controller, the main body having a radiation hole (312) formed at one side thereof, for radiating UV of the UV lamp (320) to an outside, and a through hole (314) formed at an upper portion where the switch of the controller is positioned and communicating with the outside,
wherein the main body (310) is configured to be attachable and detachable in the storage spaces of the first and second cases (10, 20).

16. The sterilizer of claim 12, wherein the UV generating means (30) comprises a single ended electronic discharge tube On/Off circuit.

17. The sterilizer of claim 12, wherein the placing means (40) comprises:
a placing bar (410); and
a hinge (420) protruded from both sides of a lower portion of the placing bar (410),
wherein any one of the first and second cases (10, 20) comprises a hinge member (19) formed at a bottom thereof, for supporting a hinge (420) of the placing bar (410).

18. The sterilizer of claim 12, wherein the placing means (40a) comprises a placing bar (410), a hinge (420) protruded from both sides of a lower portion of the placing bar (410), a rotational member (432) rotatably supporting the hinge (420), and sliding means (430) having a sliding member (434) formed at a lower portion of the placing means (40a), the placing bar (410), the hinge (420) and the rotational member (432) being formed at an upper portion of the placing means (40a).

19. The sterilizer of claim 12, wherein the placing means comprises a placing bar (510) having a gripping recess (512) formed a predetermined depth in a downward direction of a center of an upper portion, and a coupling protrusion protruded from a lower portion of the placing bar (510),
wherein any one of the first and second cases (10, 20) comprises at least one insertion hole (19b) formed at a bottom thereof, for fixedly coupling the coupling protrusion.

20. The sterilizer of claim 12, wherein the placing means comprises a placing bar (510) having a gripping recess (512) formed a predetermined depth in a downward direction of a center of an upper portion and a storage recess (524) formed a predetermined depth at a lower portion thereof, and sliding means (430) having a protrusion piece partially inserted into the storage recess (524) of the placing bar (520) and protrusion parts formed at the storage recess (524), coupled to one another to be rotatable by a hinge, and formed at a predetermined interval at a lower portion of the placing bar 520, wherein any one of the first and second cases (10, 20) comprises a sliding slot (19a) into which an interval between the placing bar (520) and the protrusion parts is inserted.

21. A sterilizer for baby products, comprising:
first and second cases (10, 20) each having a storage space therein and a hinge member (12, 22) formed at one edge thereof and into which a shaft is inserted such that the first and second cases (10, 20) are rotatably opened and closed around a shaft;
UV generating means provided at any one of the storage spaces of the first and second cases (10, 20);
placing means provided in the storage spaces of the first and second cases (10, 20) to fix the baby products; and
a fixing member (610) having at least one coupling hole (612) formed at any one of the first and second cases (10, 20) and communicating with the storage space, for fixing a neck of a bottle through the coupling hole (612) and sterilizing an inside of the bottle of which the neck is fixed.

22. The sterilizer of claim 21, wherein any one of the first and second cases (10, 20) comprises a pushing protrusion (23) for pushing a control switch of the UV generating means 30.

23. The sterilizer of claim 21, wherein the first and second cases (10, 20) comprise a plurality of drain holes for communicating with the external air and draining moisture.

24. The sterilizer of claim 21, wherein the UV generating means (30) comprises:
a UV lamp (320);
a controller controlling operations of a power supply, a switch determining whether the first and second cases (10, 20) are opened or closed, and the UV lamp (320) using a power of the power supply depending on the operation of the switch; and
a main body including the UV lamp and the controller, the main body having a radiation hole (312) formed at one side thereof, for radiating UV of the UV lamp (320) to an outside, and a through hole (314) formed at an upper portion where the switch of the controller is positioned and communicating with the outside,
wherein the main body (310) is configured to be attachable and detachable in the storage spaces of the first and second cases (10, 20).

25. The sterilizer of claim 21, wherein the placing means (40) comprises:
a placing bar (410); and
a hinge (420) protruded from both sides of a lower portion of the placing bar (410),
wherein any one of the first and second cases (10, 20) comprises a hinge member (19) formed at a bottom thereof, for supporting a hinge (420) of the placing bar (410).

26. The sterilizer of claim 21, wherein the placing means (40a) comprises a placing bar (410), a hinge (420) protruded from both sides of a lower portion of the placing bar (410), a rotational member (432) rotatably supporting the hinge (420), and sliding means (430) having a sliding member (434) formed at a lower portion of the placing means (40a), the placing bar (410), the hinge (420) and the rotational member (432) being formed at an upper portion of the placing means (40a).

27. The sterilizer of claim 21, wherein the placing means comprises a placing bar (510) having a gripping recess (512) formed a predetermined depth in a downward direction of a center of an upper portion, and a coupling protrusion protruded from a lower portion of the placing bar (510),
wherein any one of the first and second cases (10, 20) comprises at least one insertion hole (19b) formed at a bottom thereof, for fixedly coupling the coupling protrusion.

28. The sterilizer of claim 21, wherein the placing means comprises a placing bar (510) having a gripping recess (512) formed a predetermined depth in a downward direction of a center of an upper portion and a storage recess (524) formed a predetermined depth at a lower portion thereof, and sliding means (430) having a protrusion piece partially inserted into the storage recess (524) of the placing bar (520) and protrusion parts formed at the storage recess (524), coupled to one another to be rotatable by a hinge, and formed at a predetermined interval at a lower portion of the placing bar 520,
wherein any one of the first and second cases (10, 20) comprises a sliding slot (19a) into which an interval between the placing bar (520) and the protrusion parts is inserted.
